(19) United States
(12) Patent Application Publication
(10) Pub. No.: US 2018/0185171 A1
(43) Pub. Date: Jul. 5, 2018

(54) TEXTILE PIECE WITH INTERGRATED PROTHESIS FOR WOMAN

(71) Applicant: NINE SISTER COLLECTIONS, S.A. DE C.V. (MX/MX), Monterrey, Nuevo Leon (MX)

(72) Inventor: Guadalupe Del Rosario Sifuentes Lumbreras, Nuevo Leon (MX)

(21) Appl. No.: 15/737,750

(22) PCT Filed: Jul. 15, 2015
(86) PCT No.: PCT/MX2016/000005

§ 371 Date: Dec. 18, 2017

(30) Foreign Application Priority Data
Jul. 15, 2015 (MX) MU/U/2015/000375

(51) Int. Cl. A61F 2/50; A41B 11/00; A61F 2/78

(52) U.S. Cl. CPC A61F 2/50; A41B 11/00; A61F 2002/5001; A41B 2400/38; A41B 2400/32; A61F 2/78

(57) ABSTRACT

Disclosed is a utility model relating to a textile piece with a built-in prosthesis, essentially characterized in that is has a vertically arranged oval-shaped pad element which defines a concentric cavity, said cavity serving as a seat for a cup that attaches perfectly to the inner area thereof; the pad element being sewn onto the outer wall of a first stocking once the cup has been coupled to said pad element. In order for the first stocking to receive the pad element and the cup, adjustments are carried out by sewing once said pieces have been coupled together and a second stocking is attached by sewing in order to finish the assembly and to obtain a single piece which has a bulky shape and can be used as an item of clothing for women, for daily use, in order to add volume to women's calves.

### CROSS-REFERENCE TO RELATED APPLICATIONS

This application claims priority to, and is the National Stage of International Application No. PCT/MX2016/000005 filed on Jul. 15, 2015, the contents of which are incorporated by reference in their entirety.

### STATEMENT REGARDING FEDERALLY SPONSORED RESEARCH OR DEVELOPMENT

None

### Brief Summary of the Invention

The invention, refers to a garment with integrated prosthesis, such as stockings, pantyhose, socks, leggings, and stretchy pants, with the final purpose of helping people with leg problems specifically when they have extremely thin calves or any congenital defect, with suffering from varicose veins or loss in muscular mass, this visually allows correction of any problem previously mentioned. Secondary it is an attractive product for women, since it increases their self-esteem and personal security.

### FIELD OF THE INVENTION

The technical sector corresponds to the textile area, specifically to garments for women with thin calves.

### BACKGROUND OF THE INVENTION

Today there are many different styles of mesh stockings that bring support to the legs and also offer comfort, its use is usually pleasant due to the color diversity, others exists which are used for compression and to avoid muscular pains in legs partially amputated.

Quoting the most relevant documents that belong to the technical state which would be similar to the patent in question, such as document WO 2004006696 which describes a set of stockings with auto fasting without the hip area but instead with a ring of lateral bands that widen until forming one individual stocking for each leg, its function is to maintain freedom around the hip area. ES1078747 mentions a corrector refill for inferior garments such as leggings, elastic pants adjusted of gel refill, silicone or tissue whose elements are independent and dismountable allowing for interchangeable corrector refills. ES1078064 mentions a compression sock characterized by applying a gradually ascendant containment pressure. ES2366398 mentions a sock for use in sport activities that presents in the achilles tendon area a padding which is formed by two horizontal pads which are curved in the direction to the heel.

The new stocking with integrated prosthesis resolves the problem of lack of corporal mass in the calves' area due to extreme thinness, in difference to the products found in the technical state, this stocking can be utilized daily as a garment for women, it is essentially made of a construction of multiple layers, it is composed of four parts and the final presentation is its finalized assembly. Its technical characteristics are as follows:

The first element is a stocking pad made out of lycra, assembling to it an oval pad made out of foam with a curved shape that in its concave part is assembled a piece of gel material with a rounded shape that adds volume, the pieces are sown together to the first element, and finally a second stocking is added that is sown to its superior part to end up as one single piece.

### BRIEF DESCRIPTION OF THE SEVERAL VIEWS OF THE DRAWINGS

For a more complete understanding of the features and advantages of the present invention, reference is now made to the detailed description of the invention along with the accompanying figures in which:

FIG. 1 is a lateral view of the first stocking before assembling (1).

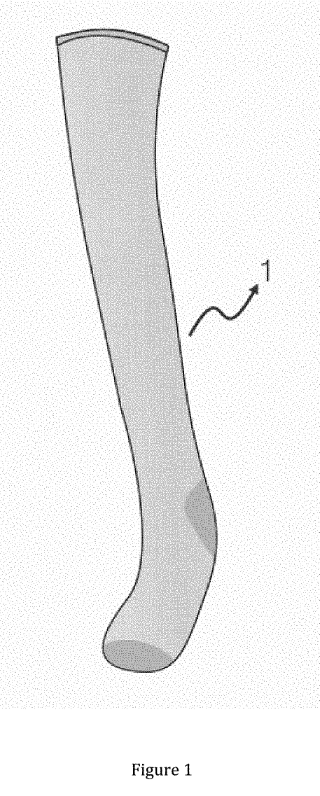

FIG. 2 is a perspective view of the oval pad made out of foam with a curved shape (2), where you can appreciate its rounded form in its superior part (3) and a better shape in a inferior part (4).

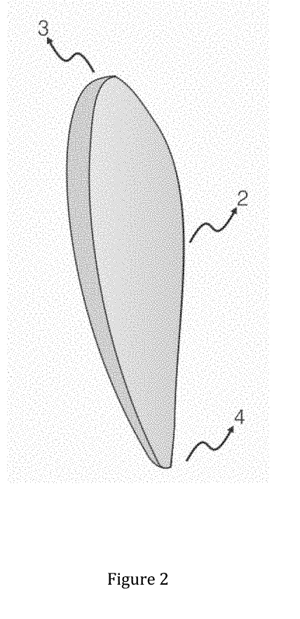

FIG. 3 is a perspective view of the pad where you can appreciate its concave central cavity (5).

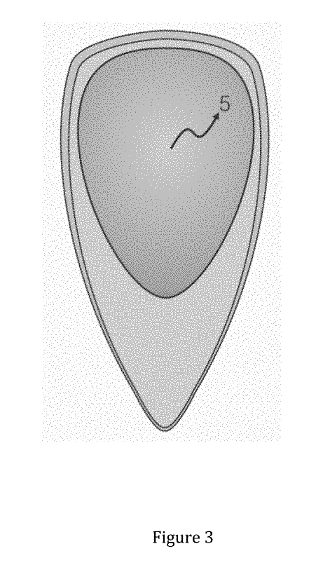

FIG. 4 is a perspective view of the cup (6) where you can appreciate its shape in accordance to the concave central cavity (5) of the pad (2).

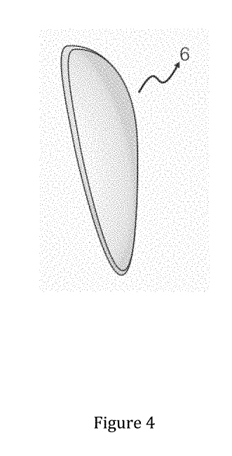

FIG. 5 is a posterior view of the cup (6).

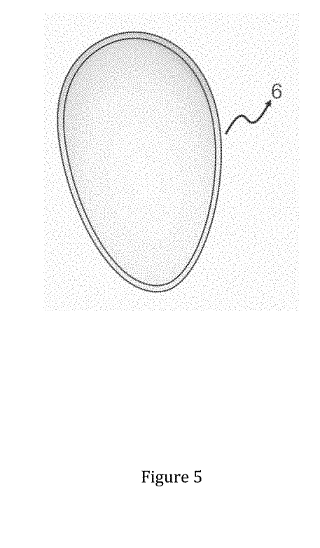

FIG. 6 is a lateral view of the second stocking (7), where you can observe the corresponding form to the suture in its superior part (8).

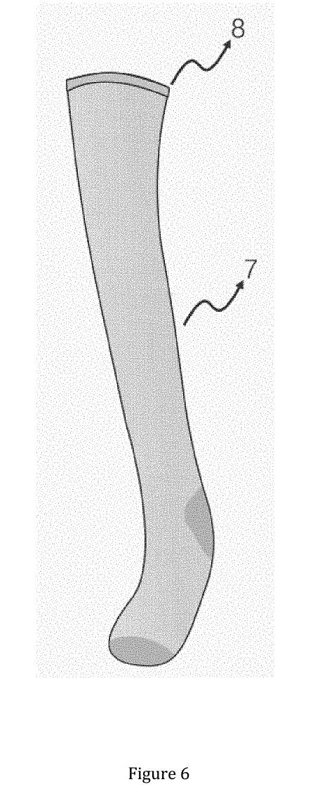

FIG. 7 is an exploded view of the union of the first stocking (1), the cup (6), the pad (2) and the second stocking (7) in which you can appreciate how each one of the elements connect.

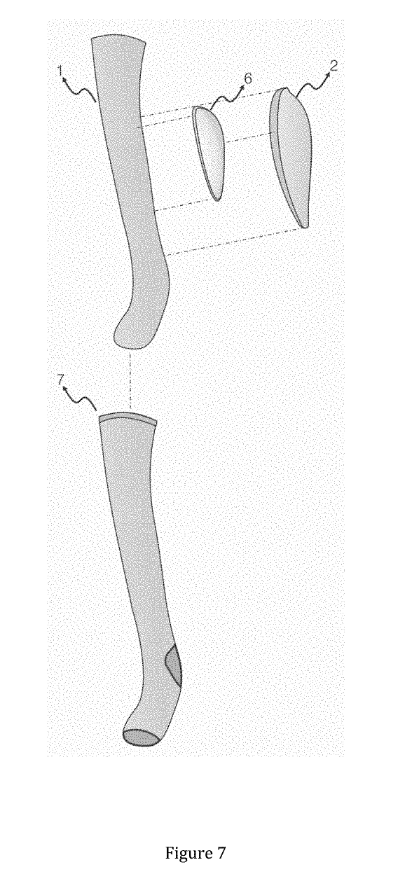

FIG. 8 is a lateral section view of the finalized piece, where you can observe the presence of the first stocking (1), the pad (2), the cup (6), and the second stocking (7), you can also observe the volume caused by the effect of the cup (6) and the pad (2).

FIG. 9 is the posterior view of the finalized piece and, where you can appreciate the first stocking (1), the pad (2), the cup (6), and the second stocking (7).

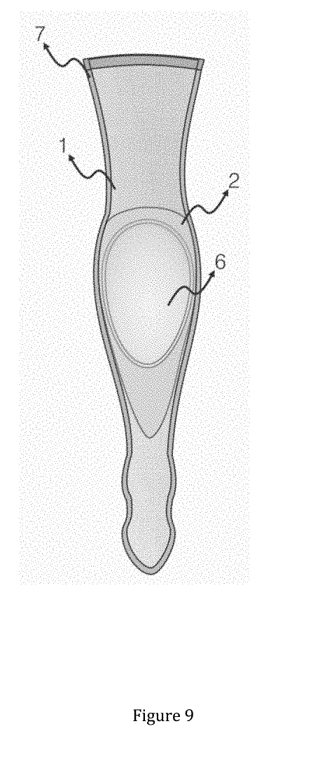

FIG. 10 is the lateral view of the finalized assembly where you can appreciate the characteristics and the volume caused by the cup and the pad (2) and (6).

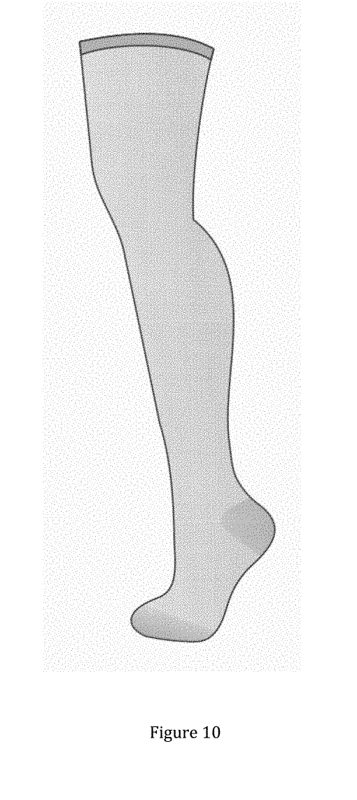

FIG. 11 is the posterior view of the finalized assembly.

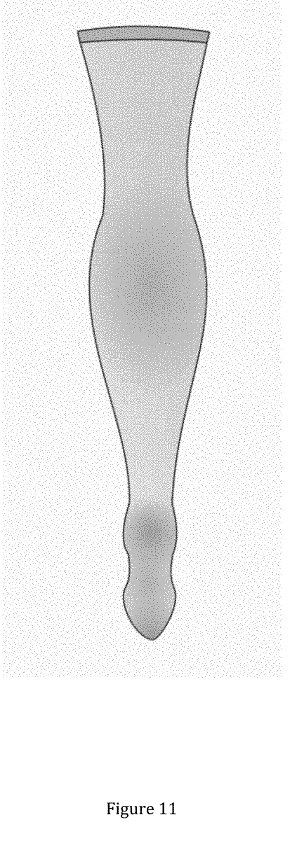

FIG. 12 is the frontal section view of the finalized assembly.

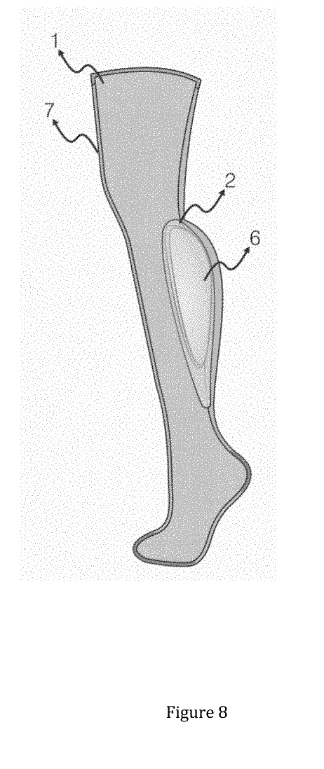

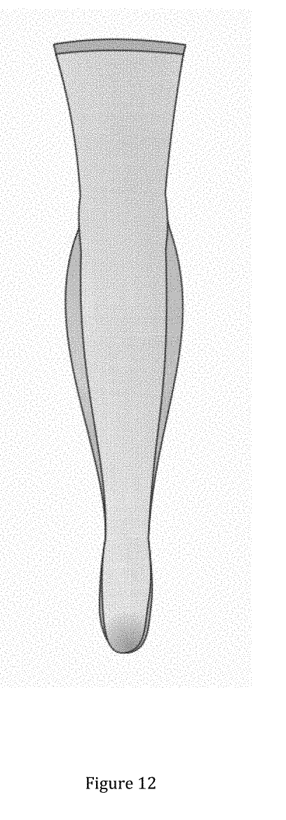

Referencing such figures you can visualize that the utility model is comprised by a stocking or textile mesh (1) which a pad (2) in a oval shape which presents a rounded end in its superior part (3) and a fading (4) in its shape in the inferior part giving a measured worn out to the material to hide the border from the pad (2) and the leg, the pad (2) is arranged vertically and sown to the piece (1) after being assembled and sown in its concentric cavity (5), the cup (6) which corresponds to a textile filling or of a soft and smooth material, which presents a rounded and bulky form, that perfectly fits in its internal area, finally the second stocking (7) is assembled, which will cover the first stocking (1), the pad (2), the cup (6) making the assembly finalized as it is appreciated in FIG. 8 so once united they form one single piece.

### DETAILED DESCRIPTION OF THE INVENTION

The following invention refers to a garment with integrated prosthesis, which could be stockings, pantyhose, socks, leggings, and stretchy pants, that is essentially characterized by being an assembly formed by a first stocking (1), a pad (2), a cup (6) and a second stocking (7). The assembly is carried out by fitting and sowing the cup and the pad to the external wall of the first stocking, and finally the second stocking is sown to the first stocking in its superior part. The first stocking is a piece of a textile material such as nontransparent mesh, which can be in various colors.

The pad is a piece made of foam with an oval shape arranged vertically that defines a concentric cavity.

The cup is a piece of textile material or soft material that can be in various sizes always in accordance to the size of the concentric cavity of the pad.

The second stocking is a piece of textile material or soft material that can be in various sizes always in accordance to the size of the concentric cavity of the pad.

The pad (2), comes with a concentric cavity (5) that functions as a seat for the cup (6) that fits perfectly in the internal area and is sown together; over the exterior wall of the first stocking (1) the pad (2) is sown after having fitted the cup (6), then the second stocking is assembled wrapping the first stocking (1), the cup (6) and the pad (2). The pad (2) presents in its superior end an oval border (3) and its inferior end presents a pointed border and also worn out lateral border (4). The first stocking (1) accommodates the pad (2), where (2) is fitted with its concave area to (6) and is adjusted to the first stocking (1) once the pieces are fitted together (1) and (2) with (6) they are fitted to the second stocking (7) to finalize the assembly and have one single textile piece with integrated prosthesis, that presents a bulky form and has the function of a woman's garment for daily use to provide volume to feminine calves with this being in the preference of women trying to better their appearance and covering their necessity of feeling safe and beautiful.

Having sufficiently described my invention, I consider it novelty and therefore claim as my exclusive property, what is contained in the following claims:

1. Textile piece with integrated prosthesis characterized by four non-dismountable parts. A total assembly in its final presentation, which contains a first stocking, a pad, a cup, and a second stocking which are sown together obtaining one single piece; the first stocking accommodates the pad in its exterior wall which fits with the concave area to the cup, once the cup, the pad, and the first stocking are put together the second stocking is added to finalize the assembly and get one single textile piece with integrated prosthesis with a bulky shape. The position of the elements during the assembly determines the perfect adjustment of the garment to the user, the stretchiness resistance without flaw, deformation or movement of the prosthesis in its use as a stocking, pantyhose, socks, leggings, and stretchy pants.

2. Textile piece with integrated prosthesis in accordance to claim 1 characterized because the first stocking is a textile piece in the form of mesh, and non transparent tissue that allows the fixation of the pad, the cup, and the second stocking, determining the characteristics of fastening of the final product to the leg of the user.

3. Textile piece with integrated prosthesis in accordance to claim 1 characterized because the pad corresponds to an oval textile piece made of foam with a curved shape, arranged vertically to the first stocking, the superior extremity is presented with an oval border and the inferior extremity presents a pointed border, also worn out on its lateral borders a characteristic that determines the borders to be unnoticeable to plain sight, with a concentric cavity in its internal part, such piece allows the accommodation over its cup cavity. Determining the fastening characteristics of the finalized product to the users' leg.

4. Textile piece with integrated prosthesis in accordance to claim 1 characterized because the cup that corresponds to a piece of textile material or soft and smooth, that is defined by its bulky shape and with its dimension according to the cavities dimension of the pad where it is assembled and sown together, determining the dimension of the characteristics of the fixation of the finalized product to the users leg.

5. Textile piece with integrated prosthesis in accordance to claim 1 characterized because the second stocking which corresponds to a textile piece of a mesh form from nontransparent tissue, that covers and allows fixation from the first stocking, the pad, and the cup making the assembly finalized, so that sowing them together forms one single piece, determining the fastening characteristics of the finalized product to the users leg.

6. The use of the textile piece with integrated prosthesis in accordance to claim 1 for the fabrication of stockings, pantyhose, socks, leggings, and stretchy pants as a finalized assembled product into one single piece, determining the fastening characteristics of the finalized product to the users' leg. Being able to use the stockings, pantyhose, and socks, with a dress do to the characteristics of its superior extremity that is presented with an oval shaped border and the inferior extremity that is presented by a pointed shape border and worn out lateral borders characteristic that determines the borders to be unnoticeable to plain sight.
